(19) United States
(12) Patent Application Publication
(10) Pub. No.: US 2022/0136071 A1
(43) Pub. Date: May 5, 2022

(54) METHODS AND SYSTEMS FOR DETECTING PATHOGENIC MICROBES IN A PATIENT

(71) Applicant: Fluent Biosciences Inc., Watertown, MA (US)

(72) Inventor: Robert Meltzer, Belmont, MA (US)

(21) Appl. No.: 17/518,129

(22) Filed: Nov. 3, 2021

Related U.S. Application Data
(60) Provisional application No. 63/109,037, filed on Nov. 3, 2020.

(51) Int. Cl. C12Q 1/6888; C12Q 1/686

(52) U.S. Cl. CPC C12Q 1/6888; C12Q 1/686

(57) ABSTRACT

This invention releases to systems and methods for detecting the presence, and preferably sequence, of microbial nucleic acids from a patient sample using droplets. In particular, methods involve probing patient nucleic acid samples with capture probes that include nucleotide sequences that are highly specific to microbial nucleic acids. Complementary microbial nucleic acids present in the sample binds to the capture probes, inside droplets, and are amplified into amplicons that can be readily detected. Samples positive for microbial nucleic acids may be sequenced to identify the microbe.

### TECHNICAL FIELD

This disclosure relates to methods and systems for detecting a microbe in a patient.

### BACKGROUND

Sepsis is a serious life-threatening illness caused by the body's response to an infection. The infection is often caused by a microbe. Antimicrobial therapy is therefore a cornerstone of sepsis treatment. Current guidelines recommend starting antimicrobial therapy within one hour of identification of sepsis. However, unless the identity of the microbe is known, finding an effective antimicrobial therapy may require trial-and-error. This approach delays effective treatment. And every hour of delay is associated with a rise in mortality. As such, early microbial detection and prompt initiation of an effective antimicrobial therapy is paramount for a positive treatment outcome.

One approach for quickly identifying a microbe is by sequencing cell-free nucleic acids. For example, cell-free nucleic acids can he taken from a patient by blood biopsy and sequenced to reveal the presence and identity of the microbe inside the patient. A treatment effective for killing the identified microbe can then be administered. Unfortunately, sequencing is generally expensive. Making such approaches unavailable to many patients.

Moreover, sequencing generally requires amplification of nucleic acids. Unfortunately, the amount of microbial nucleic acids present in a blood biopsy sample is generally low, and at low concentrations, methods of nucleic acid amplification are sensitive to losses during sample preparation and are prone to amplification biases, which may cause microbial nucleic acids to go undetected. These drawbacks inhibit the identification of pathogenic microbes, which results in delayed treatments and high mortality.

### SUMMARY

This invention provides cost-effective systems and methods for rapidly identifying nucleic acids, including but not limited to pathogenic microbes (e.g., bacteria) from patient samples. Methods include approaches for detecting the presence, and preferably sequence, of nucleic acids present in low concentrations in a sample using droplets. In particular, methods of the invention involve assaying samples with capture probes that include nucleotide sequences that are highly specific to target nucleic acids (e.g., DNA or RNA). Complementary nucleic acids present in the sample binds to the capture probes and are amplified into amplicons that can be readily detected. Because the amplicons are copies of the target nucleic acids in the sample, detection of the amplicons reveals the presence of target nucleic acids. Samples positive for target nucleic acids may be sequenced to identify the source (i.e., cell, microbe, etc.). In the event that the target is a microbe, the sequence information is useful to determine or screen treatments. Samples negative for target nucleic acids do not need to be sequenced. Detecting and sequencing only positive samples reduces the number of samples that are sequenced, thereby reducing sequencing and bioiriformatics costs. Reduced costs can increase the availability of sequencing-based treatment approaches. Additionally, methods of the invention are high throughput. For example, with a single experiment, clinicians can identify a pathogenic microbe infecting a patient and promptly administer an effective treatment. Target nucleic acid may be derived from a microbe, such as a bacterium, fungus, or virus; or may be derived from pathogenic cells, such as cancer cells, and may be derived from fetal DNA in maternal blood or host DNA in organ transplant patient.

Methods of the invention also provide approaches to faithfully amplify small amounts of nucleic acids without material loss or amplification biases. In particular, methods of the invention use emulsions to isolate, capture, and clonally amplify nucleic acids molecules inside droplets. Preferably, methods of the invention are performed with particles that template the formation of droplets inside a tube and segregate individual nucleic acid molecules therein, such that each droplet contains a single template particle and a single amplifiable nucleic acid molecule. As such, each droplet may function as an isolated reaction chamber, thereby ensuring that every nucleic acid molecule has equal access to resources required for amplification. Thus, amplification biases are significantly reduced or eliminated. Moreover, methods of the invention collect and amplify nucleic acid molecules in a single reaction tube, eliminating the need to transfer the material across multiple tubes, which prevents or eliminates material loss.

In one aspect, the invention provides a method to detect a microbial nucleic acid in a patient sample. The sample may be a blood or plasma sample with the microbial nucleic acid therein. Preferably the microbial nucleic acid comprises cell-free DNA, and more preferably, the microbial nucleic acid is cell-free 16S rDNA. The sample may also include other nucleic acids that are not from a microbe, such as, nucleic acids released from the patient's own cells. To isolate the microbial nucleic acid from other nucleic acids, methods of the invention include partitioning the sample to form a plurality of droplets simultaneously in a vessel, wherein the microbial nucleic acid is segregated inside one of the droplets. The method further includes binding the microbial nucleic acid with a capture probe inside the droplet, the capture probe includes a nucleotide sequence that is complementary to the microbial nucleic acid. The bound microbial nucleic acid is subsequently amplified to create an amplicon. The amplicon is detected, thereby detecting the microbial nucleic acid present in the sample.

In preferred embodiments, the microbial nucleic acid is associated with a 16s rDNA gene. The 16s rDNA gene is present in all known microbes and contains a favorable mix of highly conserved regions and hypervariable regions. A gene with those characteristics can be used to identify an unknown microorganism by comparing sequence reads to sequences from the same gene from known microorganisms (e.g., by aligning to those known sequences and identifying disparities). Accordingly, the microbial nucleic acid is preferably associated with the 16s rDNA gene and can thus be used to detect presence of microbial nucleic acid inside a patient sample and then be sequenced to determine the identity of the microbe.

In preferred embodiments, amplifying is performed by PCR in the presence of a fluorophore to thereby create an amplicon with the fluorophore incorporated therein. The fluorophore may include, for example, fluorescently labeled nucleotides or an intercalating dye. During amplification, the fluorophore is incorporated into the amplicon, which allows the resultant amplicon to be easily detected by, for example, measuring for a fluorescent signal from the fluorophore. As such, a sample processed by methods of the invention can be quickly assessed to determine whether the sample contains microbial nucleic acids. For example, the sample may be observed underneath a fluorescent light or device, such as a fluorometer. Amplicons present in the sample emit a fluorescent signal on account of the fluorophores. Because the amplicons are copies of microbial nucleic acids, the fluorescent signal is indicative of microbial nucleic acids present in the sample. Fluorescence labeling may occur by, for example, using an intercalating dye, fluorescence-labeled reverse primers in solution that get attached to the PlPs by a polymerization reaction, or use of sequence-specific probes (e.g., Taqman probes or molecular beacons)

Methods of the invention use droplets to capture and amplify microbial nucleic acids while eliminating interference from non-microbial nucleic acids, thereby preventing amplification biases. This is particularly useful when microbial nucleic acids are present at very small quantities, e.g., as low as 0.01% frequency of total nucleic acids in a given sample. The droplets may be prepared as emulsions, e.g., as an aqueous phase fluid dispersed in an immiscible phase carrier fluid (e.g., a fluorocarbon oil, silicone oil, or a hydrocarbon oil) or vice versa. Generally, the droplets are formed by shearing two liquid phases. Preferably, the droplets are templated by particles, referred to as template particles. Accordingly, in preferred embodiments, methods of the invention involve combining template particles with the sample in a first fluid adding a second fluid that is immiscible with the first fluid to create a mixture and vortexing the mixture to thereby partitioning the sample and form the plurality of droplets. The template particles template the formation of the droplets and segregate microbial nucleic, acid therein.

In preferred embodiments, the template particle comprises the capture probes. The capture probes may be tethered to the template particle and comprises a nucleotide sequence that is complementary to one or more portions of a 16s rDNA gene. Capture probes may also comprise a template-specific barcode, which can provide molecular identity for each amplicon as the amplicon is propagated onto the template particle through multiple rounds of PCR amplification. The template particle may comprise a plurality of capture probes with nucleotide sequences that are complementary to different portions of 16s rDNA gene, thereby allowing sequences from across a significant portion of the 16s rDNA gene to be captured and profiled.

Template particles according to aspects of the invention may comprise hydrogel, for example, selected from agarose, alginate, a polyethylene glycol (PEG), a polyacrylamide (PAA), acrylate, acrylamide/bisacrylamide copolymer matrix, azide-modified PEG, poly-lysine, polyethyleneimine, and combinations thereof. In certain instances, template particles may be shaped to provide an enhanced affinity a nucleic acid. For example, the template particles may be generally spherical but the shape may contain features such as flat surfaces, craters, grooves, protrusions, and other irregularities in the spherical shape that promote an association with a nucleic such that the shape of the template particle increases the probability of templating a monodisperse droplet that contains a nucleic acid.

Methods of the invention are useful for detecting microbial nucleic acids. The microbial nucleic acids may be any one of RNA, DNA, or both. The microbial nucleic acids may comprise cell-free nucleic acids, which can be taken from blood or plasma via non-invasive procedures. In preferred embodiments, the microbial nucleic acid is at least one of cell-free 16s rDNA or cell-free 16s rRNA. And more preferably, the microbial nucleic acid is cell-free 16s rDNA, which is more stable than 16s rRNA.

Samples positively identified for microbial nucleic acids are preferably sequenced. Sequencing produces sequence reads that are useful to identify pathogenic microbes. As such, in preferred embodiments, methods include preparing microbial nucleic acids for sequencing. Preparing may involve amplifying the amplicons. The amplicons may be amplified by PCR using primers that incorporate additional primers, such as, P5 and P7 sequencing primers.

Methods of the invention provide approaches for identifying a microbe. The method may involve using a computer system comprising a processor coupled to a memory device for analyzing sequence reads obtained by sequencing microbial nucleic acids as well as sequence information from one or more references. The references may comprise sequence information from different species of microbes and/or different strains of species. Matching the sequence reads to the references can be used to identify the microbe based on similarity. Accordingly, the method may involve aligning the sequence reads to the references and determining an alignment score between the sequence reads and sequences of references of known microbes. Determining the alignment score may include calculating match scores between bases of the sequence reads and bases in the references. An alignment score above a pre-determined threshold can be used to reveal a match, and thus identify the microbe. The method may further involve providing a report that includes the identity of the microbe. Based on the identify the microbe, a physician can administer an effective treatment to the patient to kill the microbe and thus alleviate symptoms of sepsis.

### BRIEF DESCRIPTION OF DRAWINGS

FIG. 1 diagrams a method for detecting a target nucleic acid.

FIG. 2 shows a vessel containing nucleic acids and template particles before vortexing.

FIG. 3 shows a vessel containing nucleic acids and template particles inside droplets.

FIG. 4 shows an exemplary capture probe.

FIG. 5 shows a droplet with a microbial nucleic acid and a template particle.

FIG. 6 shows the droplet with microbial nucleic acid bound to a capture probe.

FIG. 7 shows an amplicon inside a droplet.

FIG. 8 shows a final sequencing product.

### DETAILED DESCRIPTION

Pathogenic microbes (i.e., microorganisms) infect patients. The body's response to the infection can cause sepsis, which may be life-threatening. Effective treatment may require knowing the identity of the microbe, e.g., the species of microbe. High throughput sequencing represents a powerful approach for identifying pathogenic microbes. For example, microbes can be identified by sequencing nucleic acids isolated from patient blood samples to reveal nucleotide sequences that correspond with specific microbial species. But this approach remains too costly to be applied to too many samples in multiplexed sequencing reactions and the bioinformatic treatment is still not trivial. Moreover, because microbial nucleic acids are present at low concentrations, high-throughput sequencing analyses are often unreliable because of amplification biases that cause over amplification of some non-microbial nucleic acids, leaving the microbial nucleic acids to go undetected. Methods of the invention overcome these limitations by using capture probes that specifically capture and amplify microbial nucleic acids inside droplets.

In particular, methods of the invention involve probing nucleic acid samples from patients using capture probes with nucleotide sequences that are specific to microbial nucleic acids. The nucleotide sequences of the capture probes are highly specific in binding to complementary microbial nucleic acids and are thus useful to determine whether microbial nucleic acid is present in a patient sample. Any complementary microbial nucleic acids present in the sample bind to the capture probes and are thus amplified into amplicons that can be readily detected. Because the amplicons are copies of the microbial nucleic acids, detection of the amplicons reveals the presence of microbial nucleic acids inside the patient sample. Samples positive for microbial nucleic acids may be sequenced to identify the species of microbe. Samples negative for microbial nucleic acids do not need to be sequenced. As such, methods of the invention are useful for identifying samples with microbial nucleic acids for sequencing analysis. This reduces the amount of sequencing performed, thereby reducing sequencing costs,

Moreover, methods of the invention provide approaches to faithfully amplify small amounts of nucleic acids without material loss or amplification biases. In particular, methods of the invention use emulsions to isolate, capture, and clonally amplify nucleic acids molecules inside droplets, Preferably, methods of the invention are performed with particles that template the formation of droplets inside a tube and segregate individual nucleic acid molecules therein, such that each droplet contains a single template particle and a single nucleic acid molecule. As such, each droplet may function as an isolated reaction chamber, thereby ensuring that every nucleic acid molecule has equal access to resources required for amplification. Thus, amplification biases are significantly reduced or eliminated. Moreover, methods of the invention collect and amplify nucleic acid molecules in a single reaction tube, eliminating the need to transfer the material across multiple tubes, which prevents or eliminates material loss.

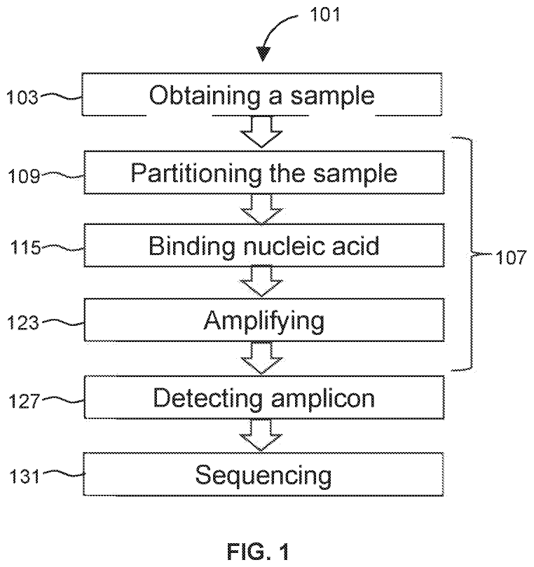

FIG. 1 diagrams a method 101 to detect a target nucleic acid. Preferably, the target nucleic acid is a microbial nucleic acid. The method 101 includes obtaining 103 a sample with a microbial nucleic acid. The sample may be a solid tissue sample or a fluid sample, such as, blood or plasma. Preferably the sample is a fluid sample. Suitable samples may include whole or parts of blood, plasma, cerebrospinal fluid, saliva, tissue aspirate, microbial culture, uncultured microorganisms, swabs, or any other suitable sample. For example, in some embodiments, a blood sample is obtained 103 (e.g., by phlebotomy) in a clinical setting. Whole blood may be used, or the blood may be spun down to isolate a component of interest from the blood, such as peripheral blood monocytes (PBMCs).

The microbial nucleic acid can be any nucleic acid useful for detecting a microbe. The nucleic acid may be RNA, DNA, or a mixture thereof. Preferably, the microbial nucleic acid comprises a cell-free nucleic acid, which is preferable because it may be taken from blood or plasma via non-invasive procedures. In preferred embodiments, the microbial nucleic acid includes at least one of cell-free 16s rDNA or cell-free 16s rRNA. And more preferably, the microbial nucleic acid is cell-free 16s rDNA, which is more stable than 16s rRNA.

In preferred embodiments, the microbial nucleic acid is associated with the 16s rDNA gene. The 16S rDNA gene (or 16S ribosomal DNA gene) is a component of the 30S small subunit of a prokaryotic ribosome that binds to the Shine-Dalgarno sequence, The genes coding for it are referred to as 16S rDNA gene and are used in reconstructing phylogenies, due to the slow rates of evolution of this region of the gene. The 16s rDNA gene is present in all known microbes and contains a favorable mix of highly conserved regions and hypervariable regions. A gene with those characteristics can be used to identify an unknown organism by comparing the sequence to sequences from the same gene from known organisms (e.g., by aligning to those known sequences and identifying disparities). Accordingly, nucleic acids associated with the 16s rDNA gene, e.g., 16s rDNA, can be used to detect presence of microbial nucleic acids inside a sample and then sequenced to determine the identity of the microbe.

Preferably, the sample is a fluid sample such as a blood sample. Obtaining 103 the sample may include performing a blood draw to obtain blood or receiving blood from a clinical facility. In some embodiments, obtaining 103 a sample involves a phlebotomy procedure and collects blood into blood collection tube such as the blood collection tube sold under the trademark VACUTAINER by BD (Franklin Lakes, N.J.) or a cell-free DNA blood collection tube such as that sold under the trademark CELL-FREE DNA BCT by Streck. Inc. (La Vista, Nev.). Any suitable collection technique or volume may be employed. A 10 ml sample of blood from a patient infected with a pathogenic microbe may contain only about 1 ng of microbial nucleic acids.

After obtaining 103 the sample, subsequent steps of the method 101 may be performed. In most instances, the sample will include nucleic acids released from the patient's own cells. These nucleic acids are not helpful for identifying the microbe and may in fact interfere with detection by obscuring the presence of microbial nucleic acids in the sample. As such, it is preferably to isolate the microbial nucleic acid away from other nucleic acids present in the sample. To isolate the microbial nucleic acid from other nucleic acids, methods of the invention include partitioning 109 the sample to form a plurality of droplets simultaneously in a vessel, wherein the microbial nucleic acid is segregated inside one of the droplets.

Partitioning 109 the sample may involve preparing an emulsion, e.g., an aqueous phase fluid dispersed in an immiscible phase carrier fluid (e.g., a fluorocarbon oil, silicone oil, or a hydrocarbon oil) or vice versa. Generally, the partitions are formed by shearing two liquid phases. Preferably, the partitions, i.e., droplets, are templated by particles referred to as template particles. Accordingly, in preferred embodiments, methods of the invention involve combining template particles with the sample in a first fluid adding a second fluid that is immiscible with the first fluid to create a mixture and vortexing the mixture to thereby partition the sample into a plurality of droplets. The template particles template the formation of the droplets arid segregate microbial nucleic acid therein.

The method 101 may include vortexing the vessel containing the sample. Vortexing is preferably done by pressing the vessel onto a vortexer, which creates sufficient shear forces inside the vessel to partition the aqueous fluid into monodisperse droplets. After vortexing, a plurality monodisperse droplets (e.g., at least 100, at least 1,000, at least 1,000,000, at least 10,000,000 or more) are formed essentially simultaneously. At least one of the droplets may have at least one microbial nucleic acid arid a template particle.

After vortexing, the method further includes binding 115 the microbial nucleic acid with a capture probe inside the droplet. The capture probe may include any fragment (usually 50-250 bases long) of DNA or RNA which can bind a complementary nucleic acid, via Watson-Crick base pairing, and also bind with at least one other material (e.g., antibody, a bead, a particle, etc.). Preferably, the capture probe is bound with one of the template particles. The capture probe includes sequences complementary to the microbial nucleic acid. Preferably, sequences are highly specific in binding to complementary 16s rDNA, which are not found in human DNA and are thus useful to determine whether microbial nucleic acid is present in the sample. In some embodiments, the capture probes include sequences that are complementary to conserved regions of 16s rDNA that are adjacent highly variable regions.

In preferred embodiments, the capture probes are attached to template particles. The capture probes may be tethered to the template particles at a 5′ end of the capture probe and comprise nucleotide sequences that are complementary to a portion of the 16s rDNA gene at a 3′ end. The template particle may comprise a plurality of distinct capture probes with nucleotide sequences that are complementary to different portions of 16s rDNA gene, thereby allowing sequences from across a significant portion of the 16s rDNA gene to be captured and profiled. To design the capture probes, one must know have sequence information for the 16s rDNA gene. Accordingly, one database useful with the present invention is Greengenes, which is a web application that provides access to 16S rDNA gene sequence alignment for browsing, blasting, probing, arid downloading. The database provides full-length small-subunit (SSU) rDNA gene sequences from public submissions of archaeal and bacterial 16S rDNA sequences. It provides taxonomic placement of unclassified environmental sequences using multiple published taxonomies for each record, multiple standard alignments, and uniform sequence-associated information curated from GenBank records. See DeSantis et al., 2006, Applied and Environmental Microbiology 72:5069-72.

Binding 115 may involve incubating the partitioned sample at a temperature between 55° Celsius and 35° Celsius for approximately 1 hour. Under these conditions, any microbial nucleic acids present in the droplets hybridize with the nucleotide sequences of the capture probes via complementary base pairing. Preferably, the microbial nucleic acid hybridizes at a 3′ end of the capture probe.

After binding 115, the microbial nucleic acid is amplified 123. Preferably, the microbial nucleic acid is amplified inside the droplet. Alternatively, the droplet may be lysed, and microbial nucleic acid bound with the template particle may be recovered and amplified. Various methods or techniques can be used to amplify 123 the microbial nucleic acid, for example, as discussed in WO 2019/139650, and WO 2017/031125, which are both incorporated by reference. Preferably, amplifying 123 is accomplished by PCR to generate a copy of the microbial nucleic acid, i.e., an amplicon.

PCR amplification involves the selective amplification of DNA or RNA targets using the polymerase chain reaction. During PCR, short single-stranded synthetic oligonucleotides or primers may be extended on a target template using repeated cycles of heat denaturation, primer annealing, and primer extension. According to some embodiments, a mixture of random synthetic primers may be included. The primers may be added to the mixture before portioning the sample. Alternatively, the primers may be stored inside a compartment on the template particle and released into the droplet via an external stimulus, such as heat. The primers bind with the microbial nucleic acid, thereby priming the microbial nucleic acid for amplification by a DNA polymerase.

In some embodiments, a primer used in an amplification reaction can be attached to a surface of a template particle. In some embodiments, a surface of the template particle can comprise a plurality of primers. In other embodiments, some primers are not attached to the template particles and rather are included in an aqueous fluid and are segregated into the monodisperse droplets upon shearing the mixture. In other embodiments, sonic primers are delivered into the droplets via compartments within the particle templates.

In some aspects, non-PCR based DNA amplification techniques may be used. For example, in some instances multiple displacement amplification (MDA) methods can be used to amplify target nucleic acids inside droplets. For example, see U.S. Pat. No. 6,124,120, which is incorporated by reference. MDA amplification may have advantages over the PCR-based methods since MDA amplification can be carried out under isothermal conditions. No thermal cycling is needed because the polymerase at the head of an elongating strand (or a compatible strand-displacement protein) will displace, and thereby make available for hybridization, the strand ahead of it. Other advantages of multiple strand displacement amplification include the ability to amplify very long nucleic acid segments (on the order of 50 kilobases) and rapid amplification of shorter segments (10 kilobases or less). In multiple strand displacement amplification, single priming events at unintended sites will not lead to artefactual amplification at these sites (since amplification at the intended site will quickly outstrip the single strand replication at the unintended site).

In some instances, amplifying 123 may occur by nonspecific amplification methods. For example, primers containing random sequences may be used. In other instances, sequence-specific amplification methods are used. Therefore, in some embodiments, amplification 123 reactions include one or more primers. For example, in some embodiments, each droplet may comprises at least 20 primer pairs. In some embodiments, each droplet may comprise at least 50 primer pairs. In some embodiment, each droplet may comprise at least 200 primer pairs. In some embodiments, each droplet may comprise at least 500 primer pairs.

In preferred embodiments, the amplifying step 123 is performed by PCR in the presence of a fluorophore in order to detect 127 the amplicon. The fluorophore may include, for example, fluorescently labeled nucleotides or an intercalating dye. Preferably, the fluorophore comprises an intercalating dye, such as, SYBR Green. During amplification, the fluorophore is incorporated into the amplicon, which allows the resultant amplicon to be easily detected 127 by measuring for a fluorescent signal from the fluorophore. As such, a sample processed by methods of the invention can be quickly assessed to determine whether the sample contains copies of target nucleic acids. For example, the sample may be observed underneath a fluorescent light or device, such as, a fluorometer. A fluorometer or fluorimeter is a device used to measure parameters of visible spectrum fluorescence: its intensity and wavelength distribution of emission spectrum after excitation by a certain spectrum of light. These parameters are used to identify the presence and the amount of specific molecules in a medium. Modern fluorometers are capable of detecting fluorescent molecule concentrations as low as 1 part per trillion. In some embodiments, the droplets are lysed to release the fluorescently labeled amplicons prior to detection. After lysing the droplets, the sample may undergo one or more washing steps to rid the sample of fluorophores not incorporated inside DNA and thus make it easier to detect the presence of amplicons. At this stage, the amplicon may still be attached to the template particle. Any amplicons present in the sample will emit a fluorescent signal on account of the tluorophores. Because the amplicons are copies of microbial nucleic acids, the fluorescent signal is indicative of microbial nucleic acids present in the sample.

Samples positive for microbial nucleic acids may be processed for sequencing 131. In some embodiments, this involves amplifying the bead-bound amplicons. Amplifying bead-bound amplicons may be performed with primers that include sequencing primers.

In some embodiments, amplified target nucleic acids may be analyzed by sequencing, which may be performed by any method known in the art. For example, see, generally, Quail, et. al., 2012. A tale of three next generation sequencing platforms: comparison of Ion Torrent, Pacific Biosciences and Illumina MiSeq sequencers, BMC Genomics 13:341. Nucleic acid sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

Sequencing generates sequence reads, which must be processed. The conventional pipeline for processing sequencing data includes generating FAST)-format files that contain reads sequenced from a next generation sequencing platform and aligning these reads to an annotated reference genome. These steps are routinely performed using known computer algorithms, which a person skilled in the art will recognize can be used for executing steps of the present invention. For example, see Kukurba, Cold Spring Harb Protoc, 2015 (11)1951-969, incorporated by reference.

The sequence reads may be analyzed to identify microbes, Various strategies for the alignment and assembly of sequence reads, including the assembly of sequence reads into contigs, are described in detail in U.S. Pat. No. 8,209,130, incorporated herein by reference, Strategies may include (i) assembling reads into contigs and aligning the contigs to a reference; (ii) aligning individual reads to the reference; or (iv) other strategies known to be developed or known in the art. Sequence assembly can be done by methods known in the art including reference-based assemblies, de novo assemblies, assembly by alignment, or combination methods. Sequence assembly is described in U.S. Pat. No. 8,165,821; 7,809,509; 6,223,128; U.S. Pub. 2011/0257889; and U.S. Pub. 2009/0318310, the contents of each of which are hereby incorporated by reference in their entirety. Sequence assembly or mapping may employ assembly steps, alignment steps, or both. Assembly can be implemented, for example, by the program The Short Sequence Assembly by k-mer search and 3′ read Extension (SSAKE), from Canada's Michael Smith Genome Sciences Centre (Vancouver, RC., CA) (see, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

The sequence reads may be aligned to one or more references to identify a microbe. Accordingly, the one or more references may include nucleotide sequences from known microbes. Matching the sequence reads from the microbial nucleic acids with the nucleotide sequences of known microbes is useful to determine the identity of the microbe in the patient based on the sequenced microbial nucleic acids. As such, in preferred embodiments, methods of the include sequencing amplicons, i.e., the copies of microbial nucleic acids from the sample, to produce a plurality of sequence reads. The amplicons may optionally be amplified prior to sequencing. Sequencing may be performed with any known sequencer.

Methods of the invention may include analyzing the sequence reads to identify the species of microbe present in the patient. Analyzing the sequence reads may include aligning them to one or more references of known microbes. This may be performed using a computer program. For example, analyzing the sequence reads may be performed with using the Basic Local Alignment Search Tool (BLAST), developed by National Center for Biotechnology information. Another database useful with the present invention is Greengenes, which is a web application that provides access to 16S rRNA gene sequence alignment for browsing, blasting, probing, and downloading.

Methods may include barcoding target fragments to prepare for downstream sequencing analysis. Any suitable methods may be used to barcode target fragments inside droplets for sequencing. Suitable approaches to attached barcodes to target fragments may include (i) fragmentation and adaptor-ligation (in which adaptors include barcodes); (ii) tagmentation (using transposase enzymes or transpososomes including those sold in kits such as those tagmentation reagent kits sold under the trademark NEXTERA by Illumina, Inc.); and (iii) amplification by, e.g., polymerase chain reaction (PCR) using primers with a hybridization portion complementary to a known or suspected target of interest in a genome and at least one barcode portion that is copied into the amplicons by the PCR reaction. For any of these approaches, the barcodes (e.g., within amplification primers or ligatable adaptors) may be provided free an in solution or bound to a template particle as described herein. In some embodiments, the barcodes are provided as a set (e.g., including thousands of copies of a barcode) in which each barcode is covalently bound to a template particle.

As used herein, barcode generally refers to an oligonucleotide that includes an identifier sequence that can be used to identify sequence reads originating from target nucleic acids that were barcoded as a set with copies of one barcode unique to that set. Barcodes generally include a known number of nucleotides in the identifier sequence between about 2 and about several dozen or more. The oligonucleotides that include the barcodes may include any other of a number of useful sequences including primer segments (e.g., designed to hybridize to a target of interest in a genetic material), universal primer binding sites, restriction sites, sequencing adaptors, sequencing instrument, index sequences, others, or combinations thereof. For example, in some embodiments, barcodes of the disclosure are provided within sequencing adaptors such as within a set of adaptors designed for use with a next generation sequencing (NGS) instrument such as the NGS instrument sold under the trademark HISEQ by Illumina, Inc. Within an NGS adaptor, the barcode may be adjacent the index portion or the target sequence such that the barcode sequence is found in the index read or the sequence read.

In some aspects, a template particle may include capture probes with portions that hybridize or ligate to microbial nucleic acid. The capture probe may include gene-specific sequences for hybridizing microbial nucleic acid by complementary base pairing. The capture probes may include a binding site sequence P5, and an index. The capture probes may further include a binding sequence P7 and a hexamer. Any suitable sequence may be used for the P5 and P7 binding sequences. For example, either or both of those may be arbitrary universal priming sequence (universal meaning that the sequence information is not specific to the naturally occurring genomic sequence being studied, but is instead suited to being amplified using a pair of cognate universal primers, by design). The index segment may be any suitable barcode or index such as may be useful in downstream information processing. It is contemplated that the P5 sequences, the P7 sequence, and the index segment may be the sequences use in NGS indexed sequences such as performed on an NOS instrument sold under the trademark ILLUMINA, and as described in Bowman, 2013, Multiplexed Illumina sequencing libraries from picogram quantities of DNA, BMC Genomics 14:466, incorporated by reference. The hexamer segments may be random hexamers or selective hexamers (aka not-so-random hexamers). Preferably, the template particles are linked to the capture oligos that include one or more primer binding sequences. However, in other aspects, the capture oligos may be released from the template particles prior to attachment with the target fragment.

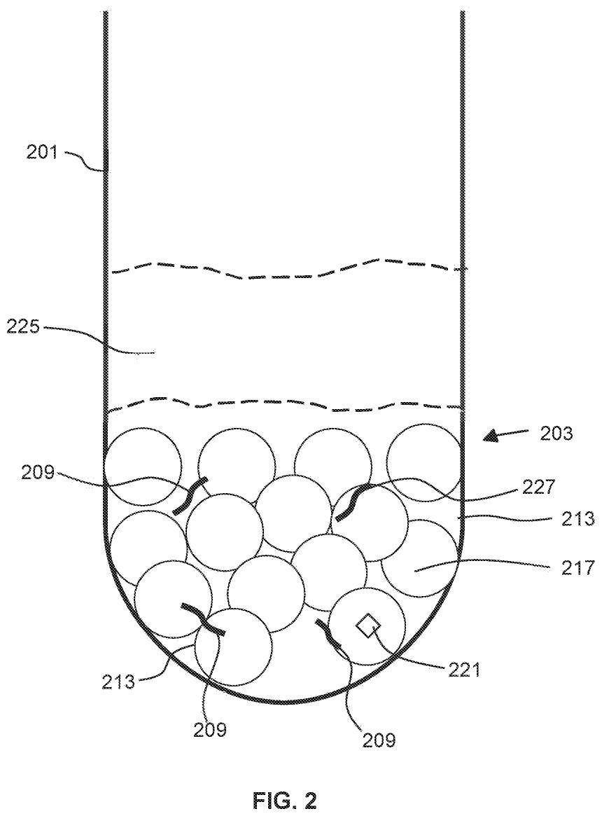

FIG. 2 shows a vessel 201 containing nucleic acid 209 and template particles 217 before vortexing. At least one of the nucleic acids is a target nucleic acid 227, e.g., a microbial nucleic acid. The vessel 201 includes a mixture of nucleic acids and template particles 217 inside an aqueous fluid 213 with an oil overlay. Shown, is an illustration of the vessel 201 after the combining step 109 of method 101. The aqueous fluid 213 may include certain reagents, such as, reagents for preserving samples of nucleic acids, e.g., EDTA, or for nucleic acid synthesis, such as, reagents for PCR. In some embodiments, the reagents may be provided by template particles 217. Accordingly, template particles 217 may include one or more compartments 221 containing the reagents, which are releasable from the compartments 221 in response to an external stimulus, such as, for example, heat, osmotic pressure, or an enzyme. Reagents may include nucleic acid synthesis reagents, such as, for example, a polymerase, primers, dNTPs, fluorophores, or buffers. In addition, the vessel 201 further includes a second fluid 225 that is immiscible with the first fluid, e.g., an oil.

In some aspects, generating the template particles-based monodisperse droplets involves shearing two liquid phases. The liquid phase comprising template particles and nucleic acids is the aqueous phase and, in some embodiments, the aqueous phase may further include reagents selected from, for example, buffers, salts, lytic enzymes (e.g. proteinase k) and/or other lytic reagents (e. g. Triton X-100, Tween-20, IGEPAL, or combinations thereof), nucleic acid synthesis reagents e.g. nucleic acid amplification reagents. The second phase is a continuous phase and may be an immiscible oil such as fluorocarbon oil, a silicone oil, or a hydrocarbon oil, or a combination thereof. In some embodiments, the fluid may comprise reagents such as surfactants (e.g. octylphenol ethoxylate and/or octylphenoxypolyethoxyethanol), reducing agents (e.g. DTT, beta mercaptoethanol, or combinations thereof). For example, see Hatori et. al., Anal. Chem., 2018 (90):9813-9820, which is incorporated by reference.

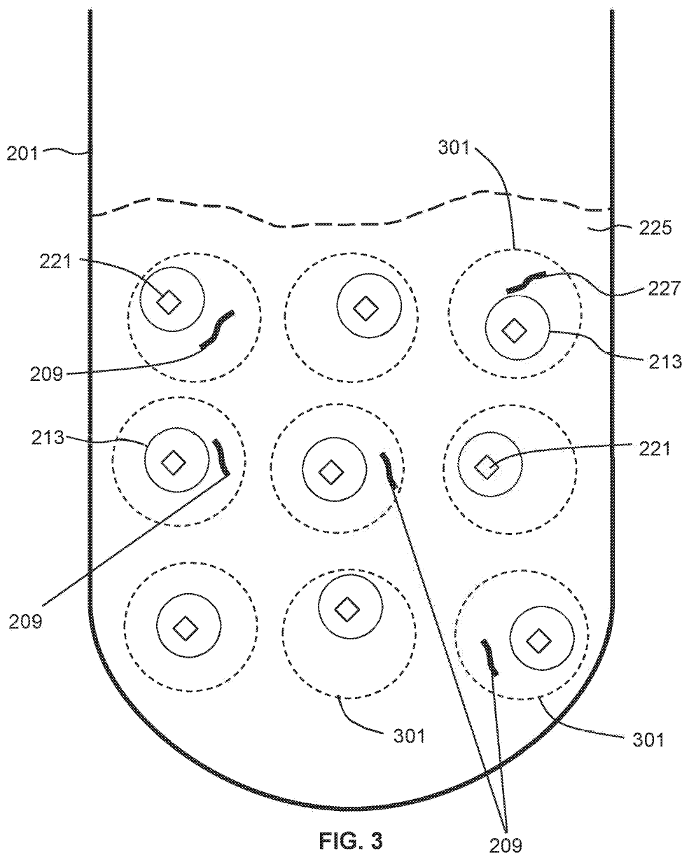

FIG. 3 shows a vessel 229 containing nucleic acids 209 and template particles 217 inside droplets. The vessel 201 includes a plurality of monodisperse droplets 301, at least one of which contains a single fragment of target nucleic acid, 227, and a temple particle 213. A person of skill in the art will recognize that not all of the droplets 301 generated according to aspects of the invention will necessarily include a single one nucleic acid and a single one of the template particles 217. In some instances, a droplet 301 may include more than one, or none, the nucleic acids or template particles 217. Droplets that do not contain one of each nucleic acid and a template particle 217 may be removed from the vessel 201, destroyed, or otherwise ignored. In some instances, template particles 217 may be formulated so as to have a positive surface charge, or an increased positive surface charge. Such materials may be without limitation poly-lysine or polyethyleneimine, or combinations thereof. This increases the probability of an association between the template particle 217 and the microbial nucleic acid, which is negatively charged.

Other strategies aimed to increase the chances of an association between nucleic acids and a template particle 217 include creating specific template particle 217 geometries. For example, in some embodiments, the template particles may have a general spherical shape but the shape may contain features such as flat surfaces, craters, grooves, protrusions, and other irregularities in the spherical shape that enhance the associate between the template particle 217 and the microbial nucleic acid thereby improving the probability that each monodisperse droplet will contain the microbial nucleic acid.

Template particles include compartments, such as, micro-compartments, or internal compartments, which may contain additional components and/or reagents, e.g., additional components and/or reagents that may be releasable into monodisperse droplets 301. Reagents may include, for example, a DNA polymerase.

Template particles of the present disclosure may include a plurality of capture probes. Generally, the capture probe is an oligonucleotide. The capture probes may be attached to the template particle's material, e.g. hydrogel material, via. covalent acrylic linkages. In some embodiments, the capture probes are acrydite-modified on their 5′ end (linker region). Generally, acrydite-modified oligonucleotides can be incorporated, stoichiometrically, into hydrogels such as polyacrylamide, using standard free radical polymerization chemistry, where the double bond in the acrydite group reacts with other activated double bond containing compounds such as acrylamide. Specifically, copolymerization of the acrydite-modified capture probes with acrylamide including a crosslinker, e.g. N,N-,methylenebis, will result in a crosslinked gel material comprising covalently attached capture probes. In some other embodiments, the capture probes comprise acrylate terminated hydrocarbon linker and combining the said capture probes with a template particle will cause their attachment to the template particle.

The capture probe may comprise one or more of a primer sequence, a barcode unique to each droplet, a unique molecule identifier (UMI), and a capture sequence.

Primer sequences may comprise a binding site, for example a primer sequence that would be expected to hybridize to a complementary sequence, if present, on any target nucleic acid molecule and provide an initiation site for a reaction, for example an elongation or polymerization reaction. The primer sequence may also be a “universal” primer sequence, i.e. a. sequence that is complimentary to nucleotide sequences that are very common for a particular set of nucleic acid fragments. The primer sequences used may be P5 and P7 primers as provided by Illumin, Inc., San Diego, Calif. The primer sequence may also allow the capture probe to bind to a solid support, such as a template particle.

By providing capture probes comprising the barcode unique to each droplet, the capture probes may be used to tag the nucleic molecules inside droplets with the barcode.

Unique molecule identifiers (UMIs) are a type of barcode that may be provided to nucleic acid molecules in a sample to make each nucleic acid molecule, together with its barcode, unique, or nearly unique. This is accomplished by adding, e.g. by ligation, one or more UMIs to the end or ends of each nucleic acid molecule such that it is unlikely that any two previously identical nucleic acid molecules, together with their UMIs, have the same sequence. By selecting an appropriate number of UMIs, every nucleic acid molecule in the sample, together with its UM, will be unique or nearly unique. One strategy for doing so is to provide to a sample of nucleic acid molecules a number of UMIs in excess of the number of starting nucleic acid molecules in the sample. By doing so, each starting nucleic molecule will be provided with different UMIs, therefore making each molecule together with its UMIs unique. However, the number of UMIs provided may be as few as the number of identical nucleic acid molecules in the original sample. For example, where no more than six nucleic acid molecules in a sample are likely to be identical, as few as six different UMIs may be provided, regardless of the number of starting nucleic acid molecules.

UMIs are advantageous in that they can be used to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may he considered to refer to the same starting nucleic acid molecule, thereby reducing amplification bias. Methods for error correction using UMIs are described in Karlsson et al., 2016, Counting Molecules in cell-free DNA and single cells RNA, Karolinska Institutet, Stockholm Sweden, incorporated herein by reference. Capture sequences used in capture probes are advantageous for targeting gene-specific nucleotide sequences, for example nucleotide sequences known to be associated with a particular cancer genotype or phenotype. In such methods, the target nucleic acid sequence, if present, attaches to the template particle by hybridizing to the capture sequence. In one aspect, this embodiment of the invention does not utilize UMIs per se. Rather, each template particle carries a unique template barcode. The capture sequence (forward PCR primer) is assembled 3′ of that barcode sequence. Reverse primers (which may be fluorescently labeled) are introduced in multiplex and free in solution. Amplification within a PIP thus requires clonal propagation of the amplified product, with all amplified product tethered to the template particle through the forward primers. Every clone, therefore, must carry the same barcode sequence. A second round of PCR with universal indexing primers releases soluble amplified primers releases soluble amplified product from the tethered amplicons.

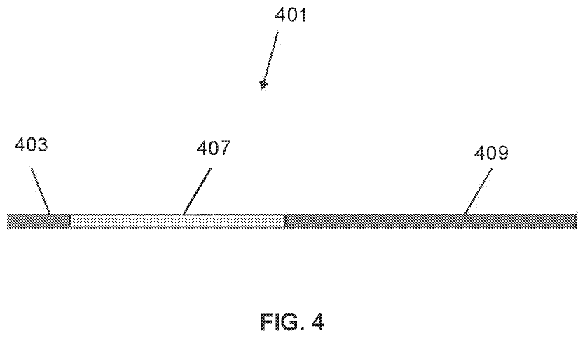

FIG. 4 shows an exemplary capture probe 401. Preferably, the capture probe 401 is attached to a template particle (not shown). The capture probe may include any number of primer binding sites and one or more barcodes. Preferably, the capture probe 401 includes a universal primer for sequencing. For example, the capture probe may include a P5 (403) or P7 primer sequence. The capture probe may further include one or more barcodes 407. The barcode may be a UMI. The capture probe 401 further includes a sequence 409 complementary to a target nucleic acid, e.g., a microbial nucleic acid. Preferably, the sequence is complementary to a portion of a 16s rDNA gene. The sequence may be complementary to a conserved portion of a 16s rDNA gene, i.e., a sequence that is conserved across a multitude of different microbial species. Preferably, the conserved portion is adjacent to a highly variable portion (i.e., a microbe specific sequence) of the 16s rDNA gene, thereby allowing the microbe to be identified after sequencing.

FIGS. 5-7 illustrate a method of preparing microbial nucleic, acids for sequencing according to aspects of the invention.

FIG. 5 shows a droplet 505 with a target nucleic acid (e.g., a microbial nucleic acid) 509 and a template particle 511. With reference to FIG. 1, the droplet 505 is preferably formed by making an emulsion with template particles 511 and nucleic acids, including the target acid 509, inside a vessel. Shown, is a single representative droplet 505 with a template particle 511 and target nucleic acid 509; although, the vessel could contain hundreds to millions of droplets. The template particle 511 includes a plurality of capture probes 401, for example, as described in FIG. 4. The capture probes 401 are tethered to the template particle 511. The capture probes include sequences complementary to the target nucleic acid 509. Accordingly, under conditions that favor hybridization, the target nucleic acid 509 binds to the capture probe 401 via complementary base pairing.

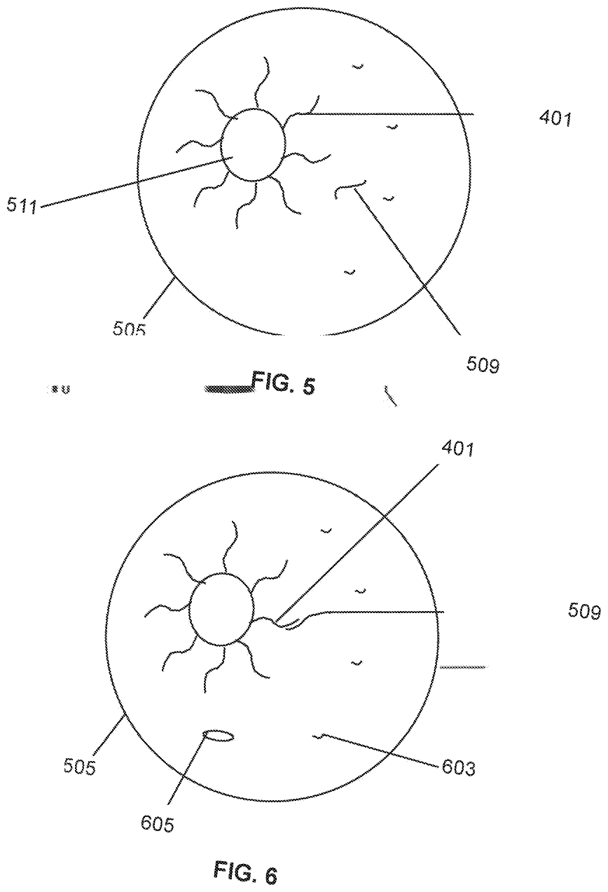

FIG. 6 shows the droplet 505 with target nucleic acid 509 bound to a capture probe 401. Shown, is the droplet 505 of FIG. 5, at a second time point, after the target nucleic acid 509 has hybridized to the capture probe 401. After hybridization, the target, nucleic acid is amplified by, for example, PCR. Preferably, amplification is performed in the presence of a fluorophore 605. A fluorophore is a fluorescent chemical compound that can re-emit light upon light excitation. Preferably, the fluorophore 605 is incorporated into an amplicon, which is made by copying the bound microbial nucleic with a polymerase, e.g., a DNA polymerase or reverse transcriptase. The presence of the fluorophore allows the amplicon to be detected. Primers 603 for PCR may be included in the mixture. The primers 603 may comprise random oligo for binding to the bound target nucleic acid.

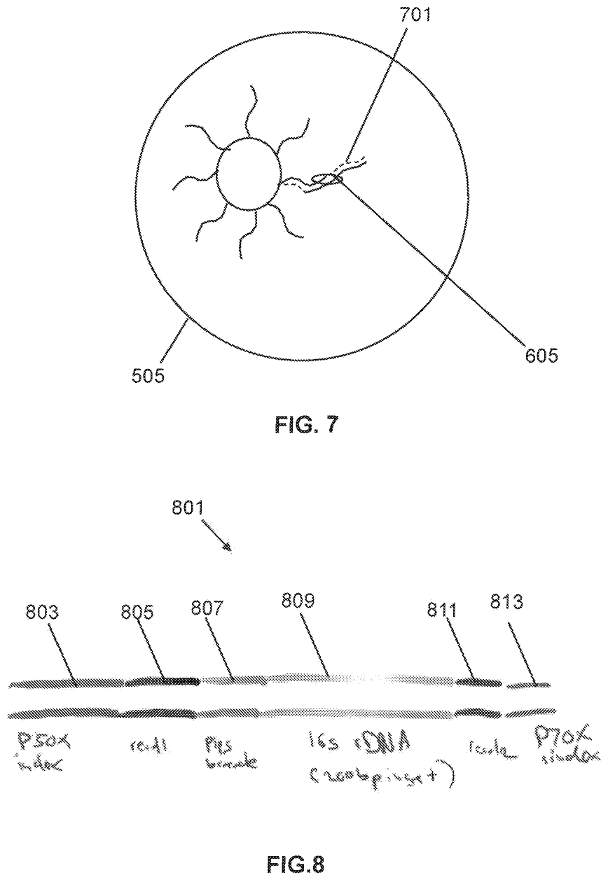

FIG. 7 shows an amplicon 701 inside a droplet 505. The amplican 701 includes the fluorophore 605 incorporated therein. The droplet may be lysed to release the template particles bound with capture probes comprising the amplicons 701. A fluorometer may then be used to detect amplicons, and as such, detect the microbial nucleic acids present in the sample based on fluorescence. In some embodiments, the final sequencing product is created by amplifying the amplicon 701 with PCR. PCR may be performed with primers for incorporating one or more additional sequencing primers into the final sequencing product.

FIG. 8 shows a final sequencing product 801. The sequencing product includes, from left to right, a P50X index 803, a Read 1 sequence 805, a barcode 807, a copy of a target nucleic acid 809, a Read 2 sequence 811, and a P70X index sequence.

The final sequencing product 801 may be sequenced to generate a plurality of sequence reads. The sequence reads may be analyzed to determine the identity of one or more microbes. Determining the identify preferably involves aligning the sequence reads with reference sequences of known microbes. To that end, a number of different databases may be helpful for obtaining reference sequences of microbes.

Ensembl Genomes is a database useful for the present invention. Ensemble Genomes provides genome-scale data from non-vertebrate species. It complements the main Ensembl database (which focuses on vertebrates and model organisms) by providing genome data for bacteria, fungi, invertebrate metazoa, plants, and protists. The bacterial division of Ensembl contains all bacterial genomes that have been completely sequenced, annotated, and submitted to the International Nucleotide Sequence Database Collaboration (European Nucleotide Archive, Gen Bank, and the DNA Database of Japan). It contains more than 15,000 genomes. Ensembl allows manipulation, analysis, and visualization of genome data. Most Ensembl Genomes data is stored in MySQL relational databases and can be accessed by the Ensembl Pearl API, virtual machines or online. See Kersey et al., 2011, Nucleic Acids Research 40:D91-97.

The DNA Data Bank of Japan (DDBJ) is another sequence database. The central DDBJ resource consists of public, open-access nucleotide sequence databases including raw sequence reads, assembly information and functional annotation. It exchanges its data with European Molecular Biology Laboratory at the European Bioinformatics Institute and with GenBank at the National Center for Biotechnology Information on a daily basis. See Kodama et al., 2012, Nucleic Acids Research 40:D38-42.

Several databases focus on a particular conserved gene, such as the 16S rDNA . For example, the EzTaxon-e database is a web-based tool for identifying prokaryotes based on 16S ribosomal RNA gene sequences. EzTaxon-e is an open access database containing sequences of type strains of prokaryotic species with validly published names. The database covers not only species within the formal nomenclatural system but also phylotypes that may represent species in nature. All sequences that are held in the EzTaxon-e database have been subjected to phylogenetic analysis, which has resulted in a complete hierarchical classification system. See Kim et al., 2012, International Journal of Systematic and Evolutionary Microbiology 62:716-21.

The Ribosomal Database Project (RDP) is another database useful with the present invention. It provides aligned and annotated rRNA gene sequence data for bacterial and archaeal small subunit rRNA genes, as well as fungal large subunit rRNA genes. RDP provides tools for analysis of high-throughput data, including both single-stranded and paired-end reads. Most tools are available as open source packages for download. See Cole et al., 2014, Nucleic Acids Research 42:D633-42.

SILVA is another database providing comprehensive, quality checked, and regularly updated datasets of aligned small (165/185, SSU) and large subunit (235/285, LSU) ribosomal RNA (rRNA) sequences for bacteria, archaea and eukarya. It has an aligner tool called SINA (SILVA INcremental Aligner) that is able to accurately align sequences based on a curated SEED alignment. The aligner determines the next related sequences using an optimized Suffix Tree server. To find the optimal alignment for a new sequence up to 40 reference sequences are taken into account. The SINA tool is not useful for typing however. See Pruesse et al., 2012, Bioinformatics 28:1823-29; and ast et al., Nucleic Acids Research 41:D590-96.

Another database useful with the present invention is Greengenes, which is a web application that provides access to 16S rRNA gene sequence alignment for browsing, blasting, probing, and downloading. The database provides full-length small-subunit (SSU) rRNA gene sequences from public submissions of archaeal and bacterial 16S rDNA sequences. It provides taxonomic placement of unclassified environmental sequences using multiple published taxonomies for each record, multiple standard alignments, and uniform sequence-associated information curated from GenBank records. See DeSantis et al., 2006, Applied and Environmental Microbiology 72:5069-72.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein.

What is claimed is:

1. A method to detect nucleic acid, the method comprising:
obtaining a sample comprising a target nucleic acid;
partitioning the sample to form a plurality of droplets simultaneously, wherein the target nucleic acid is segregated inside one of the droplets;
binding, inside the droplet, the target nucleic acid with a capture probe;
amplifying bound target nucleic acid to create an amplicon; and
detecting the amplicon to thereby detect the target nucleic acid.

2. The method of claim 1, wherein the target nucleic acid is a microbial nucleic acid.

3. The method of claim 2, wherein the amplifying comprises a polymerase chain reaction in the presence of a fluorophore, wherein said fluorophore is incorporated into the amplicon.

4. The method of claim 3, wherein the fluorophore comprises an intercalating dye.

5. The method of claim 3, wherein detecting comprises sensing a fluorescent signal from the fluorophore, wherein said fluorescent signal is indicative of the amplicon.

6. The method of claim 1, further comprising:
combining template particles with the sample in a first fluid;
adding a second fluid that is immiscible with the first fluid to create a mixture; and
vortexing the mixture, thereby partitioning the sample to form the plurality of droplets.

7. The method of claim 6, wherein the template particles template the formation of the droplets and segregate the target nucleic acid inside one of the droplets away from non-target nucleic acids present in the sample.

8. The method of claim 1, wherein the capture probe is tethered to a template particle and comprises a nucleotide sequence that is complementary to a portion of a 16s rDNA gene.

9. The method of claim 8, wherein the template particle comprises a plurality of capture probes with nucleotide sequences that are complementary to different portions of the 16s rDNA gene.

10. The method of claim 1, further comprising sequencing the amplicon to produce a plurality of sequence reads.

11. The method of claim 10, further comprising analyzing the sequence reads to characterize the target nucleic acid.

12. The method of claim 11, wherein said analyzing step comprises aligning the sequence reads to one or more references sequences.

13. The method of claim 11, wherein the target nucleic acid is derived from pathogenic bacteria.

14. The method of claim 1, wherein the sample is a blood sample.

15. The method of claim 14, wherein the target nucleic acid comprises cell-free DNA.

16. The method of claim 2, wherein the microbial nucleic acid is present in the sample at a concentration of less than 1 picogram per microliter.

17. The method of claim 2, wherein the method is performed on a subject suspected of suffering from sepsis.
